(19) United States
(12) Patent Application Publication
(10) Pub. No.: US 2023/0277822 A1
(43) Pub. Date: Sep. 7, 2023

(54) Device for Treating Uterine Bleeding and Method of Use

(71) Applicant: Avana Health, Inc., Nashville, TN (US)

(72) Inventor: John A. Morehead, Nashville, TN (US)

(73) Assignee: Avana Health, Inc., Nashville, TN (US)

(21) Appl. No.: 18/316,425

(22) Filed: May 12, 2023

Related U.S. Application Data
(63) Continuation of application No. PCT/US23/62294, filed on Feb. 9, 2023.
(60) Provisional application No. 63/267,822, filed on Feb. 10, 2022.

(51) Int. Cl. A61M 31/00; A61M 3/02; A61K 31/08; A61P 7/04

(52) U.S. Cl. CPC A61M 31/00; A61M 3/0262; A61M 3/0295; A61M 3/0291; A61M 3/0283; A61K 31/08; A61P 7/04; A61M 2205/3393; A61M 2210/1433

(57) ABSTRACT

Disclosed is a device for treating uterine bleeding, the device comprising a shaft having proximal and distal ends, with one or more lumens therethrough, a deployable sleeve at the distal end and/or an occlusive flange between the proximal and distal ends. The device is useful in a method of treating uterine bleeding comprising inserting the distal end of the device into a patient's uterus, deploying the sleeve, and/or deploying and locking the flange against the cervix, and introducing a treatment fluid through a lumen in the shaft. An example treatment fluid may be a sclerotherapeutic agent or a saline solution.

### FIELD OF THE DISCLOSURE

The present disclosure is related to the field of uterine bleeding, or obstetric hemorrhage, and devices for treating and/or quantitating the blood loss, as well as treatment methods using these devices and methods for quantitating blood loss.

### BACKGROUND

Postpartum hemorrhage (PPH) may be defined as a cumulative blood loss of greater than or equal to 1000 ml or blood loss accompanied by signs or symptoms of hypovolemia within 24 hours after birth. In a more conservative definition, PPH may also be defined as blood loss greater than 500 mL following a vaginal delivery and greater than 1000 mL after a cesarean delivery. PPH is estimated to occur in approximately 1-3% of all deliveries, where blood loss is estimated by visualization. Of note, when blood loss is measured quantitatively, the incidence of postpartum hemorrhage increases to approximately 10% of all deliveries.

Postpartum hemorrhage is the leading cause of maternal death on day of birth. Mortality is estimated at up to 20% in some areas of the world, and common morbidities include: blood transfusion, hysterectomy, thromboembolism, and postpartum anemia that requires blood or iron transfusion. The risk of mortality or severe morbidities is markedly increased in black women, including risk of disseminated intravascular coagulation (DIC) and blood transfusion. Black women are also more likely to undergo a hysterectomy for postpartum hemorrhage than non-black women.

Risk factors for postpartum hemorrhage include, but are not limited to: advanced maternal age, multiparity (having had more than one birth), genital tract injuries, operative delivery, large newborn for gestational age, and hypertensive disorders. Although the risk factors are predictive for some patients, hemorrhage can also occur in patients with no known or apparent risk factors.

Abnormal uterine bleeding (AUB), also known as atypical vaginal bleeding, is bleeding from the uterus that is abnormally frequent, lasts excessively long, is heavier than normal, or is irregular. Symptoms include vaginal bleeding that occurs irregularly, at abnormal frequency, lasts unusually long, or is more than normal. The normal frequency of menstrual periods is 22 to 38 days. Variation in the length of time between cycles is typically less than 21 days. Bleeding typically lasts less than nine days and total blood loss per period is less than 80 mL. Excessive blood loss can be defined as 80 mL or more, but may also be defined as that which negatively affects a person's quality of life. Also, bleeding more than six months after menopause can be considered abnormal uterine bleeding.

The underlying causes of AUB may include, but are not limited to: ovulation problems, fibroids, the lining of the uterus growing into the uterine wall, uterine polyps, underlying bleeding problems, side effects from birth control, and cancer. More than one category of causes may apply in an individual case. A common classification system employed in the medical field may be FIGO's PALM COEIN classification system. In this classification system, there are 9 main categories, which are included according to the acronym PALM-COEIN (pronounced “palm-koin”): polyp; adenomyosis; leiomyoma; malignancy; and hyperplasia; coagulopathy; ovulatory dysfunction; endometrial; iatrogenic; and not yet classified. In general, the components of the PALM group are discrete (i.e., structural) entities that can be diagnosed through physical or imaging techniques and/or histopathology, whereas the COEIN group is related to entities that are not defined by imaging or histopathology (i.e., non-structural).

There remains an urgent need for devices and methods to rapidly respond to and ameliorate postpartum hemorrhage and related conditions, as well as abnormal uterine bleeding.

### SUMMARY OF THE DISCLOSURE

The devices and methods disclosed herein meet these urgent needs. Thus, provided herein is a method of quickly arresting blood loss in postpartum hemorrhage and abnormal uterine bleeding (AUB). The method utilizes new devices as described herein. Advantages include at least the following: the relative risk to the patient from the treatment process itself is minimal; the devices are relatively low-cost, and could easily be shipped and stored where needed; proper use of the devices could easily be learned by any obstetrical provider; and it is expected that no long-term effects to normal uterine function would result from the treatment methods disclosed herein.

One aspect of the disclosure is directed to a method of treating post-partum bleeding, comprising the steps of 1) providing a device comprising a) a shaft having a proximal end and a distal end, where “proximal” and “distal” are from the perspective of the operator of the device; b) a first lumen enclosed within the shaft having a first port at the proximal end for introducing a treatment fluid, and a first opening at the distal end for dispensing the treatment fluid; c) a deployable sleeve at the distal end of the shaft; and d) an occlusive flange positioned between the proximal and distal ends of the shaft; 2) providing a source of treatment fluid to the first port; 3) inserting the distal end of the device into the uterus of a subject suffering from post-partum bleeding, where the post-partum bleeding is characterized by one or more bleeding blood vessels at or near a site of bleeding; 4) deploying the deployable sleeve so that the sleeve is positioned against a sidewall of the uterus and retains the treatment fluid within the uterus; 5) locking the device in place by positioning the flange against the fornix of the cervix; and 6) introducing the treatment fluid via the first port, through the first lumen, out the distal shaft opening and into the uterus, at a pressure effective to provide retrograde flow of the treatment fluid into the one or more bleeding blood vessels.

Another aspect of the disclosure is directed to a device for treating post-partum bleeding, the device comprising a) a shaft having a proximal end and a distal end; b) a first lumen enclosed within the shaft having a first port and a first valve at the proximal end operable to open or close the lumen, for introducing a treatment fluid, and a first opening at the distal end for dispensing the treatment fluid; c) a deployable sleeve at the distal end of the shaft, where the deployable sleeve is configured to insert through the cervix of a subject; and d) an occlusive flange positioned between the proximal and distal ends of the shaft. A syringe or other injection means can also serve as the first valve. The device can further comprise a second lumen within the shaft, having a second port at the proximal end and a second opening at the distal end for drainage.

Another related aspect of the disclosure is directed to a method of treating post-partum bleeding, comprising the steps of 1) inserting the distal end of the above device for treating post-partum bleeding into the uterus of a subject suffering from post-partum bleeding, comprising one or more bleeding blood vessels; 2) providing a source of treatment fluid to the first port; 3) deploying the deployable sleeve so that the sleeve is positioned against a sidewall of the uterus and retains the treatment fluid within the uterus; 4) locking the device in place by positioning the flange against the fornix of the cervix; and 5) introducing the treatment fluid via the first port, through the first lumen, out the distal shaft opening and into the uterus, at a pressure effective to provide retrograde flow of the treatment fluid into the one or more bleeding blood vessels at or near a site of bleeding.

Yet another aspect of the disclosure is directed to a method of treating abnormal uterine bleeding, comprising the steps of 1) providing a device comprising a) a shaft having a proximal end and a distal end; and b) a lumen enclosed within the shaft having a port at the proximal end for introducing a treatment fluid, and an opening at the distal end for dispensing the treatment fluid; 2) providing a source of treatment to the port; 3) inserting the distal end of the device into the external cervical os of a subject suffering from abnormal uterine bleeding, where the abnormal uterine bleeding is characterized by one or more bleeding blood vessels at or near a site of bleeding; and 4) introducing the treatment fluid via the port, through the lumen, out the distal shaft opening and into the uterus, at a pressure effective to provide retrograde flow of the treatment fluid into the one or more bleeding blood vessels. The source of the treatment fluid can be an injection means. The injection means can comprise a syringe.

Still another aspect of the disclosure is directed to a device for treating abnormal uterine bleeding, where the device comprises a) a shaft having a proximal end and a distal end; and b) a lumen enclosed within the shaft having a port at the proximal end and a valve operable to open or close the lumen, for introducing a treatment fluid, and an opening at the distal end for dispensing the treatment fluid. The device can further comprise a conical gel tip on the shaft's distal end.

A further related aspect of the disclosure is directed to a method of treating abnormal uterine bleeding, comprising the steps of 1) inserting the distal end of the above device for treating abnormal uttering bleeding into the external cervical os of a subject suffering from abnormal uterine bleeding, where the abnormal uterine bleeding is characterized by one or more bleeding blood vessels at or near a site of bleeding; 2) providing a source of treatment fluid to the port; and 3) introducing the treatment fluid via the port, through the lumen, out the distal shaft opening and into the uterus, at a pressure effective to provide retrograde flow of the treatment fluid into the one or more bleeding blood vessels.

Another aspect of the disclosure is directed to a method for determining quantitative blood loss (QBL) in obstetric hemorrhage, comprising the steps of: 1) providing a device comprising a) a shaft having a proximal end and a distal end; b) a first lumen enclosed within the shaft having a first port at the proximal end for optionally introducing a treatment fluid, and a first opening at the distal end for dispensing the optional treatment fluid; c) a second lumen within the shaft having a second port at the proximal end and a second opening at the distal end for evacuating blood therethrough; d) a deployable sleeve at the distal end of the shaft; and e) an occlusive flange positioned between the proximal and distal ends of the shaft; 2) inserting the distal end of the device into the uterus of a subject suffering from obstetric hemorrhage; 3) deploying the deployable sleeve so that the sleeve is positioned against a sidewall of the uterus and seals the uterine cavity; 4) locking the device in place by positioning the flange against the fornix of the cervix; 5) evacuating blood through the second lumen within the shaft to a device configured to measure or weight the evacuated blood; and 6) optionally, introducing a treatment fluid through the first port. The device of the QBL method can be configured to measure or weight evacuated blood, comprising a receiving container, measuring container, weighing container, receiving means, measuring means, or weighing means.

It is to be understood that both the foregoing general description and the following detailed description are exemplary and explanatory only, and are not restrictive of the disclosure as disclosed.

Further details and embodiments of the disclosure are apparent from in the drawings.

### BRIEF DESCRIPTION OF THE DRAWINGS

FIG. 1 is a perspective view of a postpartum hemorrhage treatment device corresponding to the disclosure, including an undeployed sleeve at the distal end of the shaft, and an undeployed and unpositioned occlusive flange in the middle of the shaft, with means for deploying both the sleeve and the flange.

FIG. 2 is an exploded view of the postpartum hemorrhage treatment device shown in FIG. 1, depicting the configuration of the various components.

FIG. 3A shows an exterior view of the device of FIG. 1; FIG. 3B is a cutaway view of the device, being cut away along the line shown in FIG. 3A.

FIGS. 4A-4E display various perspective views of the device of FIG. 1: 4A, side view; 4B, front view; 4C, back view; 4D, top view; and 4E, bottom view.

FIG. 5 is a perspective view of the postpartum hemorrhage treatment device shown in FIG. 1, with both the deployable sleeve and occlusive flange expanded and deployed.

FIG. 6A shows an exterior view of the deployed device of FIG. 5; FIG. 6B shows a cutaway view of the deployed device, being cut away along the line identified in FIG. 6A.

FIG. 7 is a schematic view of an aspect of a method of the disclosure illustrating the step of inserting the postpartum hemorrhage treatment device shown in FIG. 1 into a patient's postpartum uterus.

FIG. 8 is a schematic view of an aspect of a method of the disclosure illustrating the sleeve deployed against a sidewall of the interior of the postpartum uterus, and the occlusive flange deployed and positioned against the fornix of the cervix to lock the device in place.

FIG. 9 shows a cutaway and expanded view of the distal end of the undeployed device as depicted in FIG. 3B.

FIG. 10 shows a cutaway and expanded view of the distal end of the deployed device as depicted in FIG. 6B.

FIG. 11A shows a skeleton view of the undeployed sleeve; FIG. 11B shows the same undeployed view with the surrounding external cover; FIG. 11C shows a skeleton view of the sleeve deployed to form the multiple anchors; FIG. 11D shows the same deployed view including the surrounding external cover.

FIG. 12A shows a skeleton view of a section of the undeployed device comprising the undeployed sleeve and associated slidable member with hand grip; FIG. 12B shows the same undeployed view including the external cover surrounding the undeployed sleeve.

FIG. 13A shows a skeleton view of a section of the deployed device comprising the deployed sleeve and associated slidable member with hand grip; FIG. 13B shows the same deployed view including the external cover surrounding the deployed sleeve.

FIG. 14 is a perspective view of an abnormal uterine bleeding (AUB) treatment device corresponding to the disclosure, including a shaft comprising a lumen and a conical gel tip at the distal end.

FIG. 15 is an exploded view of the AUB device shown in FIG. 14, depicting the configuration of the various components.

FIGS. 16A-16C display various perspective views of the AUB treatment device of FIG. 14: 16A shows an exterior view of the device; 16B shows a cutaway view of the device along the line identified in 16A; 16C shows a detail of the cutaway view of the distal end of the device.

FIG. 17 is a schematic view of an aspect of a method of the disclosure illustrating the step of inserting the AUB treatment device shown in FIG. 14 into a patient's vagina, positioned to contact and seal the external cervical os.

FIG. 18 illustrates an initial non-deployed configuration of an alternate AUB device having a balloon.

FIG. 19 illustrates a deployed configuration of the device in FIG. 18 in which the balloon is inflated.

FIG. 20 shows an initial non-deployed configuration of an alternate PPH device having a balloon.

FIG. 21 shows the PPH device of FIG. 19 in a deployed configuration in which the balloon is inflated.

FIG. 22 shows another illustration schematically showing relative motion of a moveable handle of the device of FIGS. 20 and 21.

### DETAILED DESCRIPTION

As disclosed herein, a number of ranges of values are provided. The recitation of numerical ranges by endpoints includes all numbers and fractions subsumed within that range, as well as the recited endpoints. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither, or both limits are included in the smaller ranges is also encompassed within the disclosure, subject to any specifically excluded limit in the stated range. Where the stated range includes one or both of the limits, ranges excluding either or both of those included limits are also included in the disclosure. The term “about” generally includes up to plus or minus 10% of the indicated number. For example, “about 10%” may indicate a range of 9% to 11%, and “about 20” may mean from 18 to 22. Preferably “about” includes up to plus or minus 6% of the indicated value. Alternatively, “about” includes up to plus or minus 5% of the indicated value. Other meanings of “about” may be apparent from the context, such as rounding off, so that, for example, “about 1” may also mean from 0.5 to 1.4.

With respect to the device, the terms “proximal” and “distal” refer to the perspective of the operator of the device, with the distal end being the end inserted into the vagina, and the proximal end being closest to the operator.

Unless otherwise defined, all terms used in disclosing the disclosure, including technical and scientific terms, have the meaning as commonly understood by one of ordinary skill in the art to which this disclosure belongs.

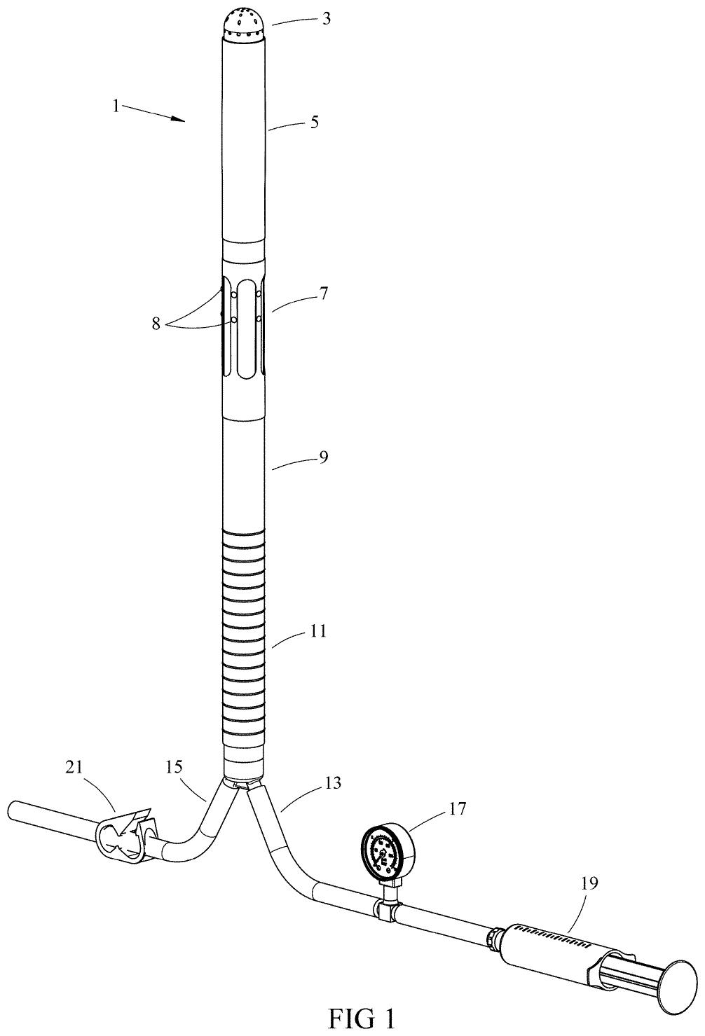

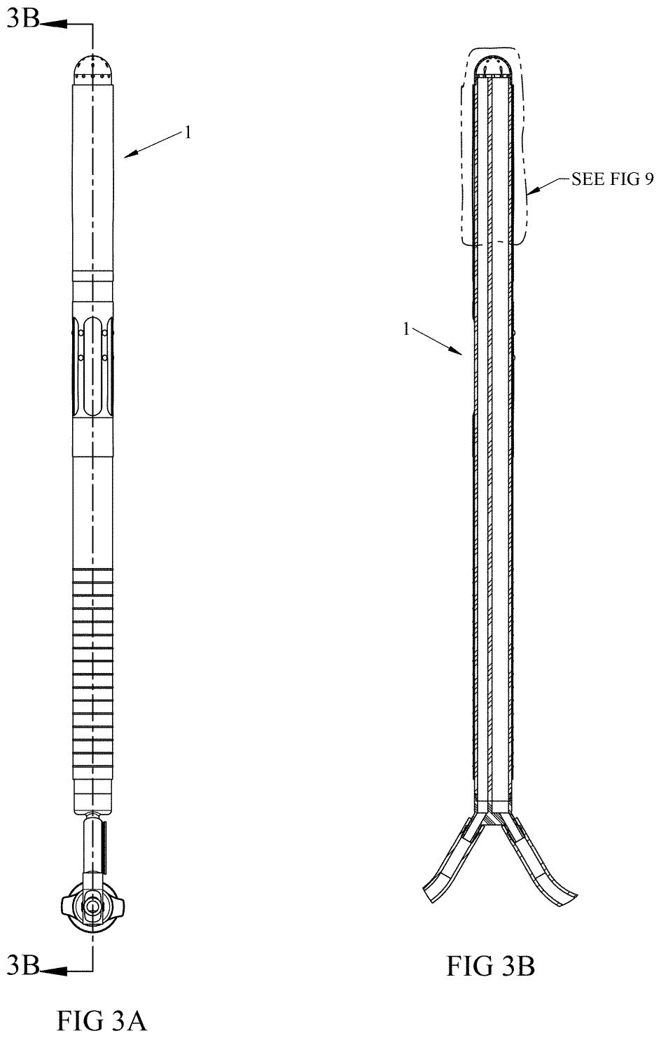

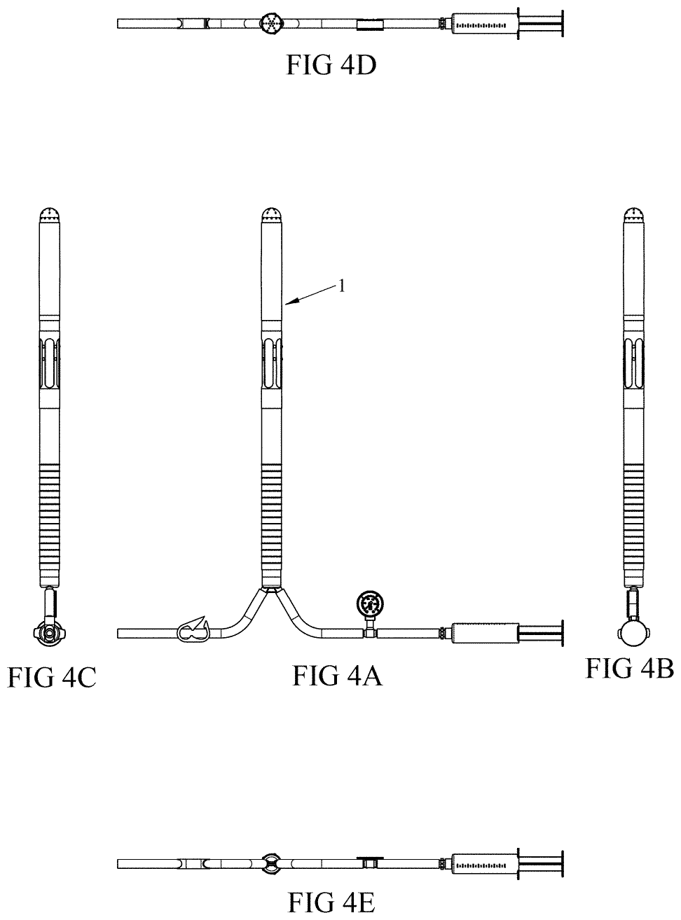

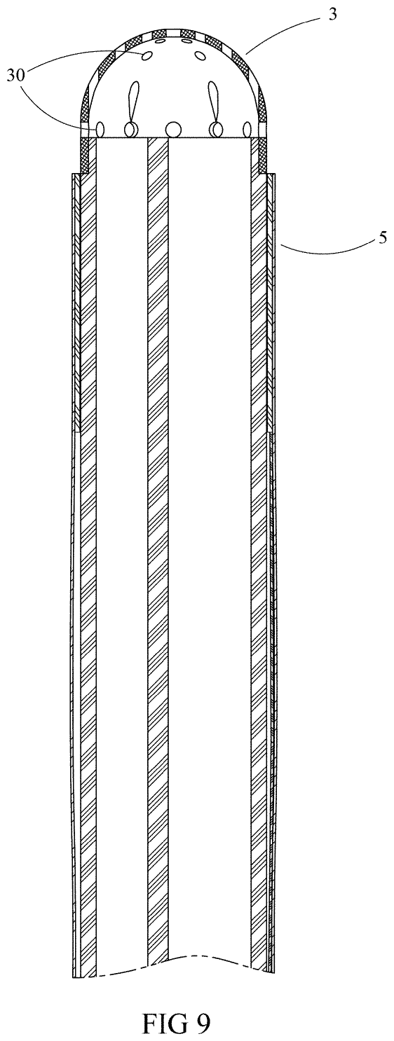

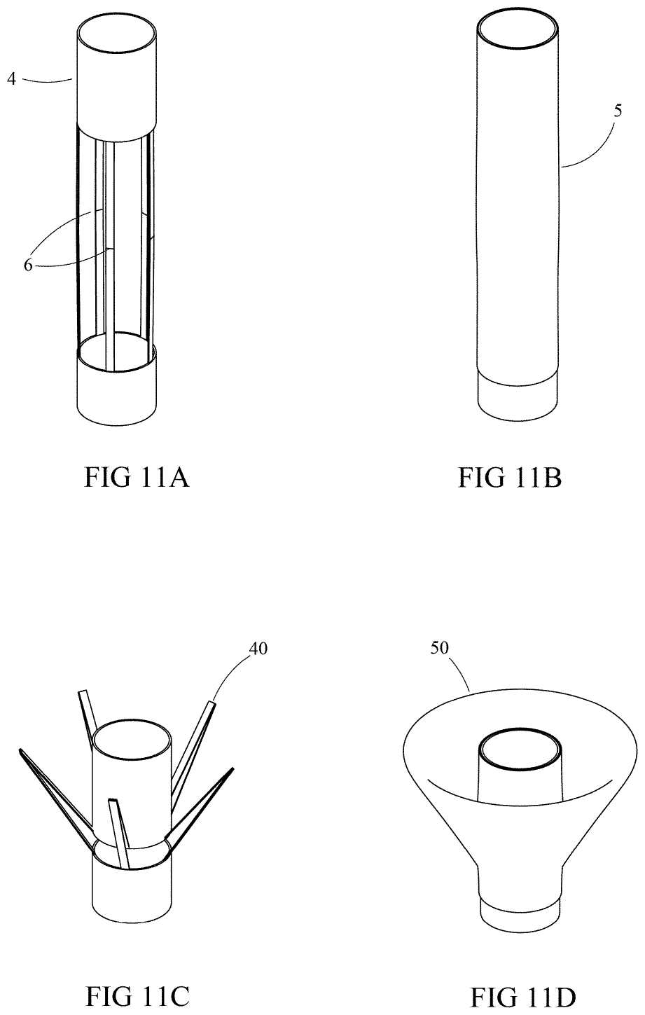

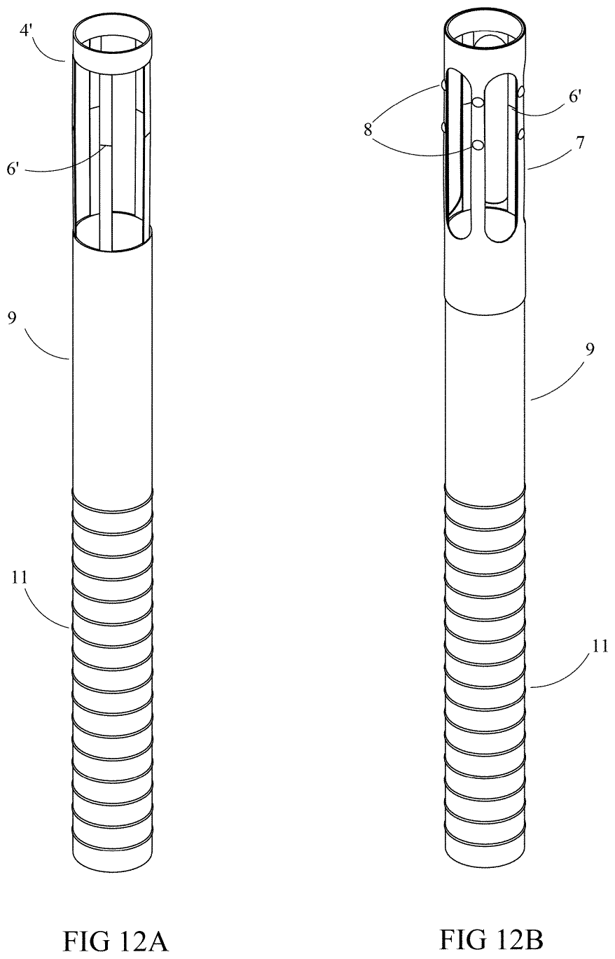

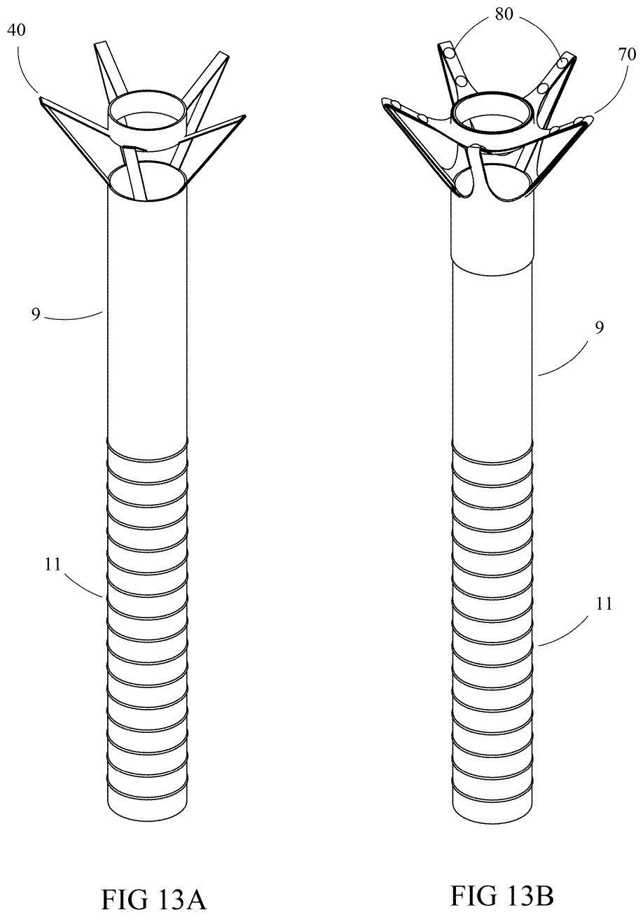

One embodiment of the inventive device is displayed in FIGS. 1 to 6 and 9 to 13. Thus, a postpartum hemorrhage treatment device 1 comprises a shaft member 2 containing one or more lumens, an endcap member 3, an externally covered undeployed sleeve member 5, an undeployed occlusive flange member 7, a slidable segment member 9, a hand grip member 11, a proximal endcap member 12 with ports to the lumens, an injection line member 13 feeding into a first lumen within the shaft, a drain line member 15 connecting to the second lumen within the shaft for drainage, a pressure gauge member 17, an injection member 19, e.g., a syringe or a bag, and a valve member 21 on the drainage line, which member can be a pinch clamp. See FIGS. 1, 2, and 4A-4E. Detailed views of members of the undeployed postpartum hemorrhage treatment device are shown in FIGS. 9, 11A, 11B, 12A and 12B. The skeleton view FIG. 11A shows the underlying skeleton framework 40 of the undeployed sleeve, minus the cover; FIG. 11B shows the undeployed sleeve plus cover. The skeleton view FIG. 12A shows the underlying framework of the undeployed occlusive flange, minus the cover; FIG. 12B shows the undeployed occlusive flange with cover. The skeleton view FIG. 11C shows the underlying framework of the deployed sleeve, minus the cover; FIG. 11D shows the deployed sleeve including cover. The skeleton view FIG. 13A shows the underlying framework of the deployed flange, minus the cover; FIG. 13B shows the deployed flange with cover.

The occlusive flange can further comprise an external cover. The external cover can comprise a soft, stretchable membrane, such as a silicone rubber membrane, which can further comprise grip pads or grip tabs 8 made of the same silicone rubber material. Alternatively, the grip pads can comprise a different rubber material. These grip pads allow the flange to contact the cervical tissue without irritating or damaging it. The multiple anchors of the flange can deploy to grip and seal the external cover against the cervix, thereby locking it in place. In one embodiment, opening and deployment results in a flange that resembles an inverted umbrella.

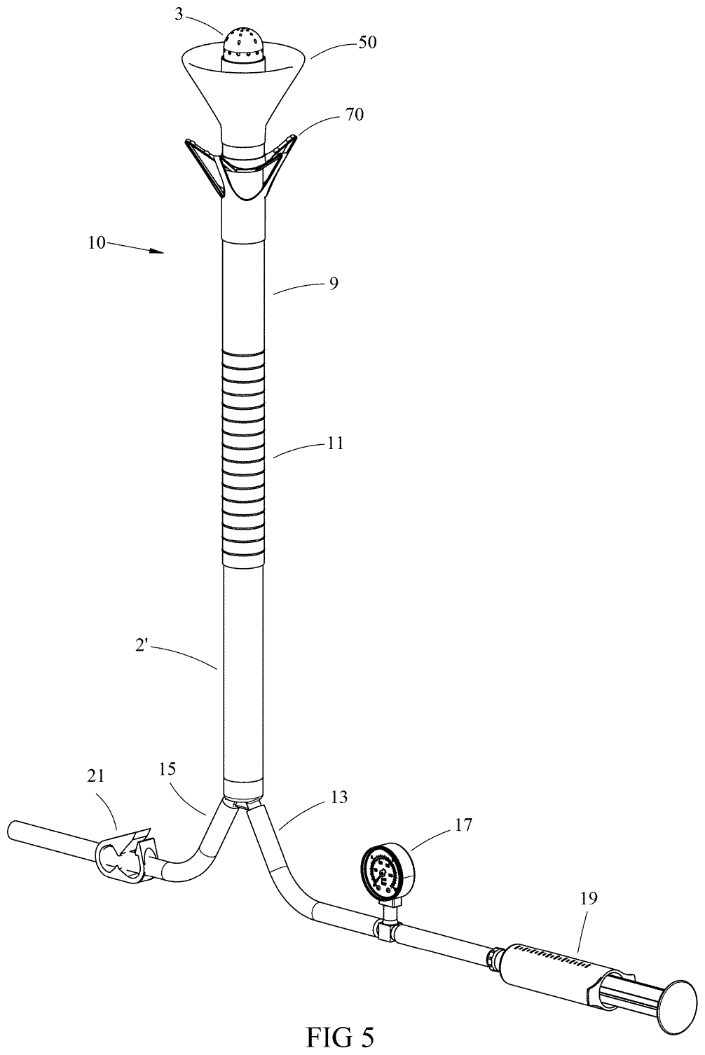

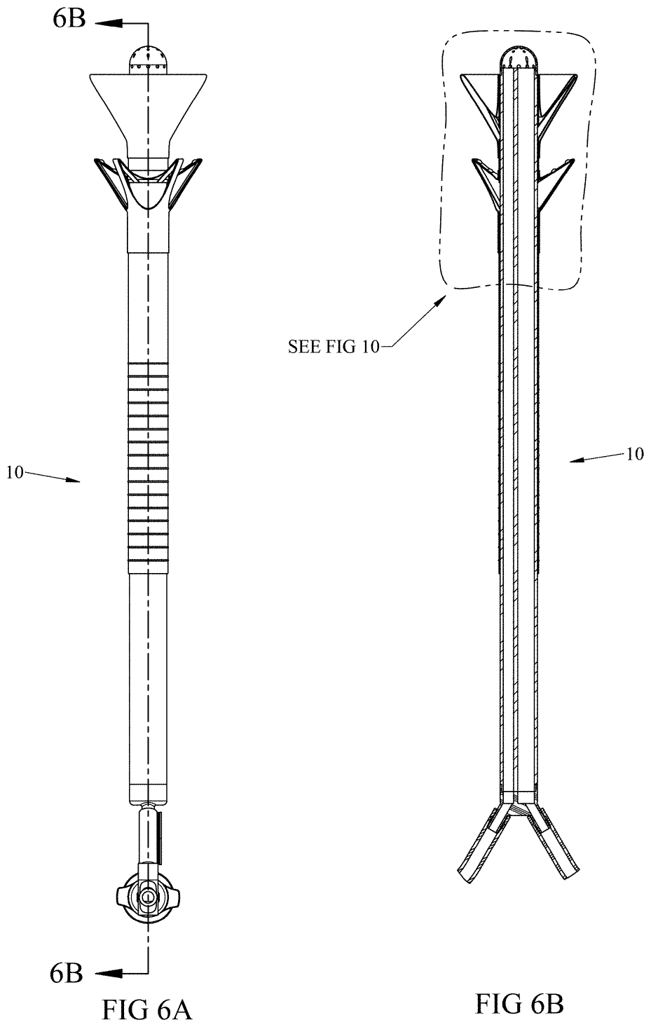

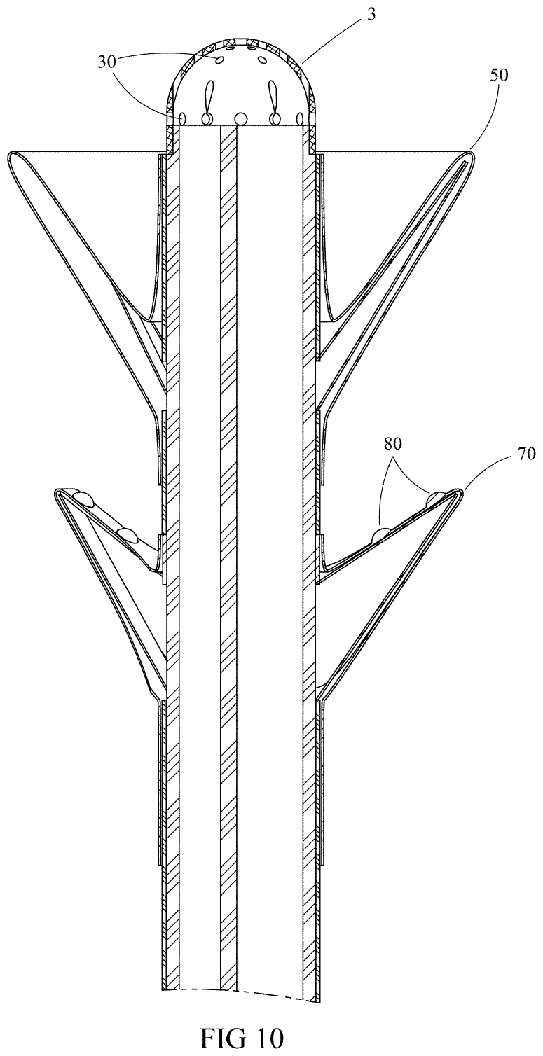

A deployed version of the device can be seen in FIGS. 5, 6A and 6B. Detailed views of members of the deployed postpartum hemorrhage treatment device 10 are shown in FIGS. 10, 11C, 11D, 13A and 13B. Note that undeployed sleeve 5 (including external cover) has been deployed to form deployed sleeve 50 (including external cover), and undeployed occlusive flange 7 (including external cover with grip pads 8) has been deployed to form the deployed occlusive flange 70 (including external cover with grip pads 80).

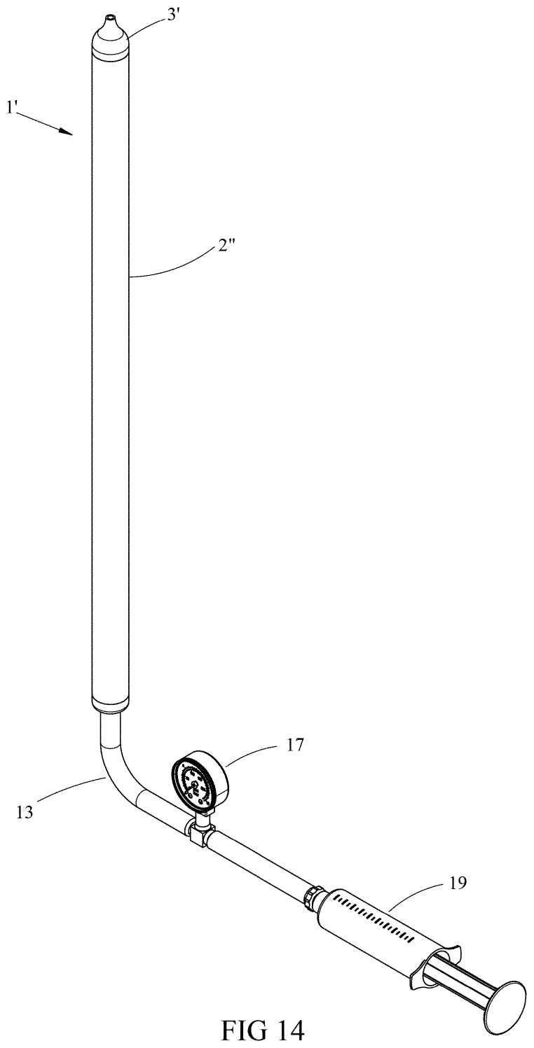

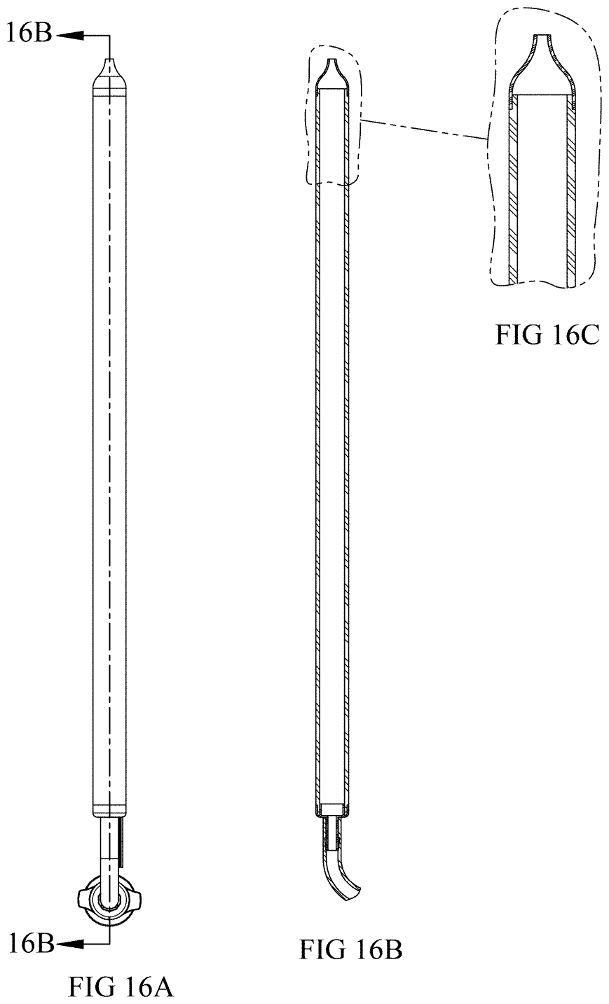

Another embodiment of the inventive device is displayed in FIGS. 14 to 16. Thus, an abnormal uterine bleeding (AUB) treatment device (non-pregnancy related) 1′ comprises a shaft member 2″ containing a single lumen, a conical gel tip member 3′, a proximal endcap member 12′ with a port to the lumen, an injection line member 13 feeding into the lumen within the shaft, a pressure gauge member 17, and an injection member 19, e.g., a syringe.

The conical shape of the tip 3′ is designed to make a seal with the external cervical os. This tip comprises an elastic gel that conforms to the external cervical os in order to provide a seal with exertion of manual pressure. The elastic gel is a moldable gel that can conform to the size and shape of the cervix to form a water-tight seal to provide a comfortable surface, and can comprise, for example, a non-latex silicone rubber.

### Features Identified in the Figures are Labelled as Follows

1 undeployed device of the disclosure
1′ another device of the disclosure
2 shaft containing one or more lumens
2′ exposed shaft after deploying the sleeve and/or occlusive flange
2″ shaft containing a single lumen
3 distal endcap
3′ distal conical gel tip
4 skeleton framework of undeployed sleeve
4′ skeleton framework of undeployed occlusive flange
5 undeployed sleeve including external cover
6 fold points in skeleton of undeployed sleeve
6′ fold points in skeleton of undeployed occlusive flange
7 undeployed occlusive flange
8 grip pads (tabs) on the undeployed occlusive flange cover
9 slidable member over the shaft
10 deployed device of the disclosure
11 hand grip on the slidable member
12 proximal endcap with ports to lumens
12′ proximal endcap with port to the single lumen
13 injection line feeding into first lumen within the shaft
15 drain line connecting to second lumen within the shaft, for drainage
17 pressure gauge
19 injection means (syringe or bag)
21 valve (pinch clamp)
30 pores or holes in distal endcap allowing outflow from the shaft lumens and inflow into the shaft lumens
40 skeleton framework of deployed occlusive flange
50 deployed sleeve including external cover
70 deployed occlusive flange, including external cover with grip pads
80 grip pads on the deployed occlusive flange cover
100 patient uterus
110 interior sidewall of uterus
120 cervix of uterus
200 AUB device having a balloon
202 shaft of device 200
203 distal endcap of device 200
212 proximal endcap of device 200
213 first injection port of device 200
214 second injection port of device 200
230 at least one lumen of device 200
260 balloon of device 200
261 hollow tube of device 200
300 PPH device having a balloon
302 interior shaft of device 300
303 distal endcap of device 300
309 moveable shaft of device 300
311 ergonomic handle of device 300
312 proximal endcap of device 300
313 first injection port of device 300
314 second injection port of device 300
315 drainage port of device 300
330 at least one lumen of device 300
350 deformable cup of device 300
360 balloon of device 300
361 hollow tube of device 300
370 flange of device 300
371 lower portion of flange 370
372 end portion of flange 370

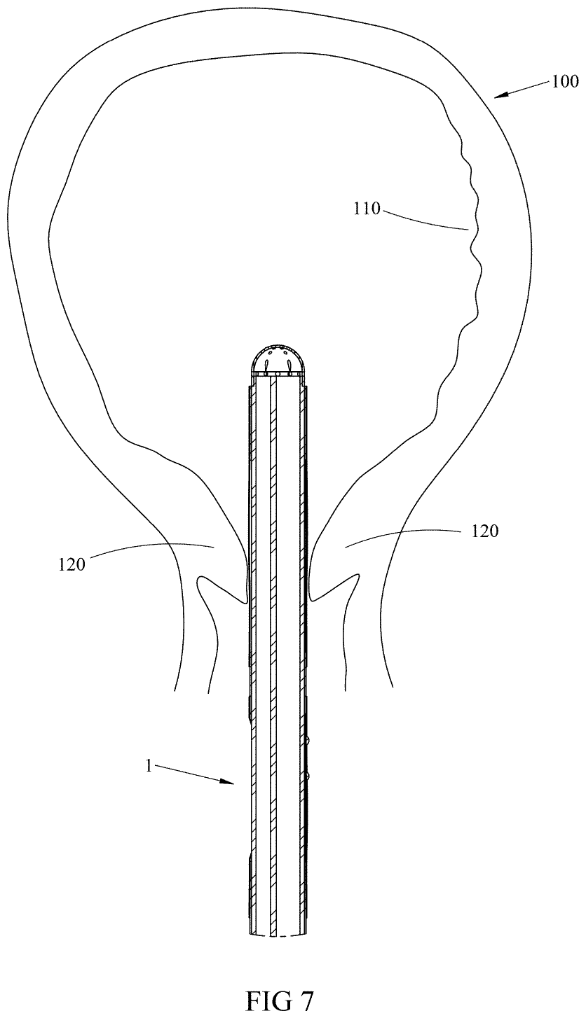

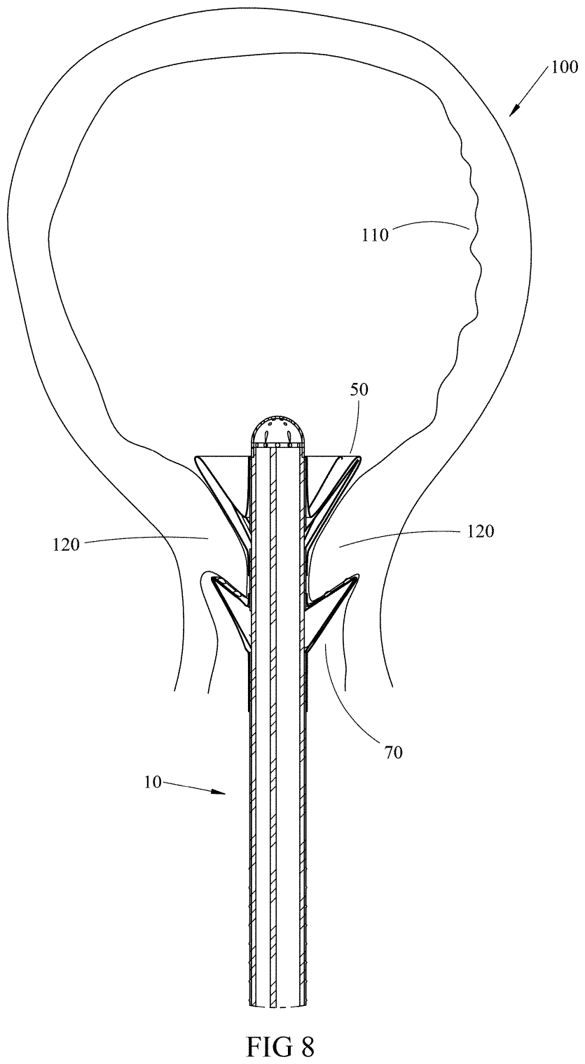

One embodiment of a method of using the devices of the disclosure is as follows. Thus, a postpartum hemorrhage treatment device corresponding to the disclosure is inserted into the vagina of a postpartum patient suffering postpartum hemorrhage and actuated as follows. The distal end of the undeployed device 1 (FIG. 1) is inserted into and positioned in the postpartum patient's bleeding uterus, as shown in FIG. 7. The deployable sleeve 5 is deployed against a sidewall of the uterus 110 forming the deployed sleeve 50 (FIG. 8), thereby sealing the interior of the uterus. Sequentially or simultaneously, the occlusive flange 7 (FIG. 1) is deployed to form deployed flange 70 (FIG. 8) and positioned against the cervix 120, thereby locking the device in place. In one embodiment, both the sleeve and flange are deployed and positioned simultaneously, for example, by movement of slidable member 9 using the hand grip member 11 toward the distal end of the device. See FIGS. 5 and 8. FIG. 5 shows the exposed shaft segment 2′ after deploying the sleeve and occlusive flange. Deployment is accomplished by folding of the undeployed 5 and 7 at the fold points 6 and 6′, respectively (see FIGS. 11A-11D, 12A and 12B, and 13A and 13B) to form 50 and 70, and locking the device in place (FIG. 8). Grip pads 80 on the deployed occlusive flange cover serve to keep the device in place without damage or irritation to sensitive cervical tissue. Thereafter, treatment solution is introduced by the injection means 19, e.g., a syringe, through the injection line 13 into the first lumen of the shaft 2 and out through the first opening at the distal end via the pores 30 in endcap 3 and into the uterus. The treatment solution is maintained under an appropriate pressure to provide retrograde flow of treatment solution into one or more bleeding blood vessels, the pressure being monitored by pressure gauge 17.

Following treatment, excess solution and any blood can be evacuated back through the pores 30 in endcap 3, into the second lumen within shaft 2, to a drain line 15 for drainage and safe waste disposal. Drainage can be spontaneous, upon release of the valve (pinch clamp 21), alternatively can be assisted by applying vacuum pressure, or both. In at least one embodiment, suction pressure is applied to drain line 15 by a suction bulb or a pump (not shown).

Preferably, grip pads are applied to the occlusive flange cover in areas where the flange contacts the cervix. These grip pads can comprise the same elastomer as the soft, stretchable membrane that covers the occlusive flange, e.g., a silicone rubber elastomer, or alternatively can comprise a different elastomer. The multiple anchors of the padded flange deploy to seal the external cover against the cervix, thereby locking it in place without causing irritation or damage to the cervical tissue. In one embodiment, opening and deployment of the covered occlusive flange resembles an inverted umbrella.

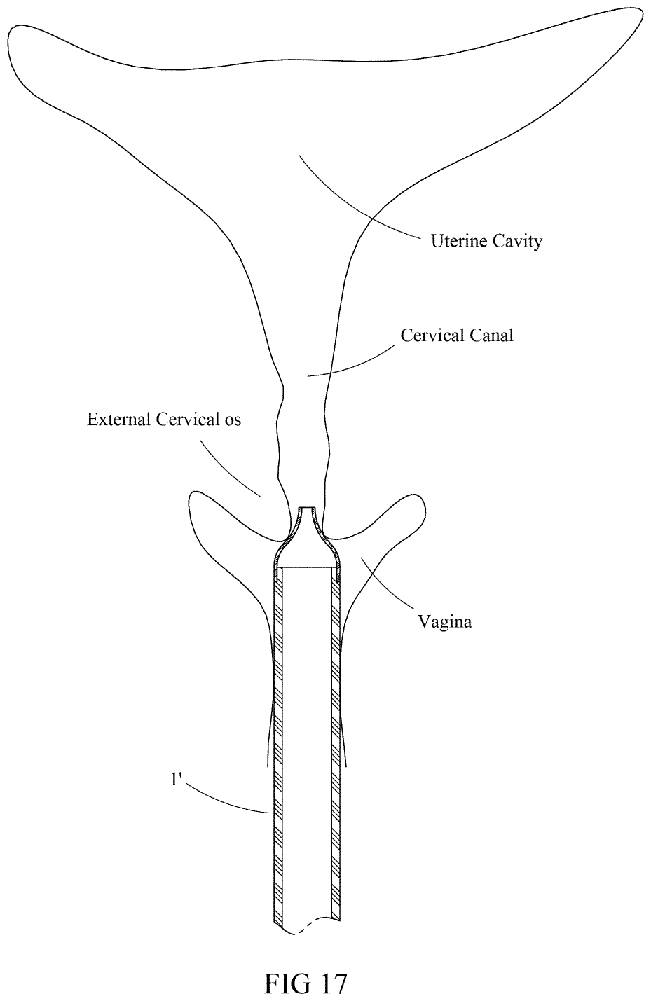

Another embodiment of a method of using the devices of the disclosure is as follows. Thus, an AUB treatment device corresponding to the disclosure is inserted into the vagina of a patient suffering from abnormal uterine bleeding (non-pregnancy related) and actuated as follows. The distal end of the device 1′ (FIG. 14) is inserted into the vagina and positioned against the patient's external cervical os, as shown in FIG. 17. Forward manual pressure from the practitioner applied through the shaft 2″ of the device seals the conical gel tip against the external cervical os to provide a seal. Thereafter, treatment solution is introduced by the injection means 19, e.g., a syringe, through the injection line 13 into the lumen of shaft 2″ and out through the conical gel tip 3′, through the cervical canal and into the uterine cavity. The treatment solution is maintained under an appropriate pressure to provide retrograde flow of solution into one or more bleeding blood vessels, the pressure being monitored by pressure gauge 17.

Following completion of treatment, the device is removed, and spontaneous drainage of the treatment solution and blood occurs.

Thus, one aspect of the disclosure is directed to a method of treating post-partum bleeding, comprising the steps of 1) providing a device comprising a) a shaft having a proximal end and a distal end; b) a first lumen enclosed within the shaft having a first port at the proximal end for introducing a treatment fluid, and a first opening at the distal end for dispensing the treatment fluid; c) a deployable sleeve at the distal end of the shaft; and d) an occlusive flange positioned between the proximal and distal ends of the shaft; 2) providing a source of treatment fluid, optionally comprising a sclerotherapeutic agent, to the first port; 3) inserting the distal end of the device into the uterus of a subject suffering from post-partum bleeding, where the post-partum bleeding is characterized by one or more bleeding blood vessels at or near a site of bleeding; 4) deploying the deployable sleeve so that the sleeve is positioned against a sidewall of the uterus and retains the treatment fluid within the uterus; 5) locking the device in place by positioning the flange against the fornix of the cervix; and 6) introducing the treatment fluid via the first port, through the first lumen, out the distal shaft opening and into the uterus, at a positive pressure effective to provide retrograde flow of the treatment fluid into the one or more bleeding blood vessels.

The source of the treatment fluid for the method of treating post-partum bleeding can be an injection means. The injection means can comprise a syringe. The deployable sleeve for the method of treating post-partum bleeding can be configured to radially expand the sleeve from the shaft when deployed. The deployable sleeve can further comprise multiple anchors that peripherally expand to seal the uterine cavity. The deployable sleeve can further comprise an external cover. The external cover can comprise a soft, stretchable membrane, such as a silicone rubber membrane. The multiple anchors of the deployable sleeve can deploy to seal the external cover within the uterine cavity. In one embodiment, opening and deployment resemble the opening of an umbrella. The deployable sleeve can be manually deployed or activated by pressure of the treatment fluid.

The occlusive flange for the method of treating post-partum bleeding can be slidable along the shaft of the device. The occlusive flange is a retention means that locks the device in place and causes the injected treatment fluid to be retained in the uterus under the desired positive pressure so that the bleeding vessels are infiltrated with the treatment solution. The flange can be ring-shaped, cone-shaped, or can comprise two arcs of a circular shape. The flange can also comprise a foldable member that folds into ribs that define a cone-shaped pattern when deployed by folding. See the undeployed segment 7 of FIG. 1, which folds into the deployed segment 70 of FIG. 5. The occlusive flange can further comprise an external cover. The external cover can comprise a soft, stretchable membrane, such as a silicone rubber membrane, which can comprise grip pads or tabs made of the same or different silicone rubber material. These grip pads allow the flange to contact and grip the cervical tissue without irritating or damaging it. The multiple anchors of the flange can deploy to seal the external cover against the cervix, thereby locking it in place. In one embodiment, opening and deployment resemble an inverted umbrella.

The device for the method of treating post-partum bleeding can further comprise a second lumen within the shaft, having a second port at the proximal end and a second opening at the distal end for drainage. The shaft can contain the second lumen within the shaft. The second lumen can be parallel to the first lumen. The second lumen can surround the first lumen.

When using the device for the method of treating post-partum bleeding, an appropriate positive intrauterine pressure should be maintained. The intrauterine pressure is preferably maintained at about 10 to about 100 mm Hg, or about 10 to about 60 mm Hg, or about 15 to about 55 mm Hg, or about 20 to about 50 mm Hg, or about 25 to about 45 mm Hg, or about 30 to about 40 mm Hg, or about 30 to about 60 mm Hg, or about 30 to about 100 mm Hg, or about 50 to about 100 mm Hg. The intrauterine pressure can be about 10, 15, 20, 25, 30, 35, 40, 45, 50, 55, 60, 65, 70, 75, 80, 85, 90, 95 or about 100 mm Hg. More preferably, an intrauterine pressure of at least about 30 mm Hg is maintained. In most cases the optimal pressure window for treatment is about 73 mm Hg to about 88 mm Hg. In some target applications, a practitioner may establish a target pressure of about 80 mm Hg with +/−8 mm Hg variation.

The first lumen of the device for the method of treating post-partum bleeding can further comprise a first valve operable to open or close the lumen. A syringe or other injection means can also serve as the first valve. The second lumen can further comprise a second valve operable to open or close the lumen. This second valve can comprise a pinch clamp.

The sclerotherapeutic agent of the method can be a vascular sclerosing agent. Sclerosants include detergents, such as sodium tetradecyl sulfate, polidocanol, sodium morrhuate and ethanolamine oleate; osmotic agents, such as hypertonic sodium chloride solution and sodium chloride solution with dextrose; and chemical irritants, such as chomated glycerin and polyiodinated iodine. The sclerotherapeutic agent preferably comprises polidocanol.

An appropriate volume of sclerotherapeutic treatment fluid is injected, depending on the patient circumstances and extent of bleeding. An appropriate volume of sclerotherapeutic fluid used may be based on the volume capacity of the particular patient's intrauterine cavity. The volume capacity for treatment varies with the particular patient's anatomy and with the state of uterine muscle tone at the time of treatment. This volume may be determined by the attending practitioner based on observation during the course treatment. For some patients, a second solution of about 0.9% Normal saline may be infused within the patient after the sclerotherapy solution has been administered in order to augment the administered volume and allow complete filling of larger capacity intrauterine cavities.

The source of the treatment fluid for the method of treating post-partum bleeding can further comprise a reservoir. The reservoir can be integral to the device, or separate and connected to it prior to dispensing the treatment fluid.

The method of treating post-partum bleeding can further comprise administering a second therapeutic agent.

Another aspect of the disclosure is directed to a device for treating post-partum bleeding, the device comprising a) a shaft having a proximal end and a distal end; b) a first lumen enclosed within the shaft having a first port and a first valve at the proximal end operable to open or close the lumen, for introducing a treatment fluid, and a first opening at the distal end for dispensing the treatment fluid; c) a deployable sleeve at the distal end of the shaft, where the deployable sleeve is configured to insert through the cervix of a subject; and d) an occlusive flange positioned between the proximal and distal ends of the shaft. A syringe or other injection means can also serve as the first valve.

The above device for treating post-partum bleeding can further contain a second lumen within the shaft. The second lumen can be parallel to the first lumen. The second lumen can surround the first lumen. The deployable sleeve can further comprise an external membrane cover. The external cover can comprise a silicone rubber membrane. The deployable sleeve can further comprise multiple anchors that peripherally expand to seal the uterine cavity. The occlusive flange can be slidable along the shaft. The above device can further comprise a second lumen within the shaft, having a second port at the proximal end, a second opening at the distal end for drainage, and a second valve. The device can further comprise a source of treatment fluid, optionally comprising a sclerotherapeutic agent, where the source provides the treatment fluid to the first port. The sclerotherapeutic agent can be a vascular sclerosing agent. The vascular sclerotherapeutic agent can include and/or be polidocanol.

A related aspect of the disclosure is directed to a method of treating post-partum bleeding, comprising the steps of 1) inserting the distal end of the above device for treating post-partum bleeding into the uterus of a subject suffering from post-partum bleeding, comprising one or more bleeding blood vessels; 2) providing a source of treatment fluid, optionally comprising a sclerotherapeutic agent, to the first port; 3) deploying the deployable sleeve so that the sleeve is positioned against a sidewall of the uterus and retains the treatment fluid within the uterus; 4) locking the device in place by positioning the flange against the fornix of the cervix; and 5) introducing the treatment fluid via the first port, through the first lumen, out the distal shaft opening and into the uterus, at a positive pressure effective to provide retrograde flow of the treatment fluid into the one or more bleeding blood vessels at or near a site of bleeding. The method can further comprise administering a second therapeutic agent.

Yet another aspect of the disclosure is directed to a method of treating abnormal uterine bleeding, comprising the steps of 1) providing a device comprising a) a shaft having a proximal end and a distal end; and b) a single lumen enclosed within the shaft having a port at the proximal end for introducing a treatment fluid, and an opening at the distal end for dispensing the treatment fluid; 2) providing a source of treatment fluid, optionally comprising a sclerotherapeutic agent, to the port; 3) inserting the distal end of the device into the external cervical os of a subject suffering from abnormal uterine bleeding, where the abnormal uterine bleeding is characterized by one or more bleeding blood vessels at or near a site of bleeding; and 4) introducing the treatment fluid via the port, through the lumen, out the distal shaft opening and into the uterus, at a positive pressure effective to provide retrograde flow of the treatment fluid into the one or more bleeding blood vessels. Preferably the distal end of the shaft comprises a conical tip designed to make a seal with the external cervical os. This conical tip preferably comprises a soft elastic gel that conforms to the external cervical os in order to provide the seal via manual pressure. The elastic gel is a moldable gel that can conform to the size and shape of the cervix to form a water-tight seal to provide a comfortable surface, and can comprise, for example, a non-latex silicone rubber. The source of the treatment fluid can be an injection means. The injection means can comprise a syringe.

The positive intrauterine pressure for method of treating abnormal uterine bleeding can be maintained at about 10 to about 100 mm Hg, or about 10 to about 60 mm Hg, or about 15 to about 55 mm Hg, or about 20 to about 50 mm Hg, or about 25 to about 45 mm Hg, or about 30 to about 40 mm Hg, or about 30 to about 60 mm Hg, or about 30 to about 100 mm Hg, or about 50 to about 100 mm Hg. The intrauterine pressure can be about 10, 15, 20, 25, 30, 35, 40, 45, 50, 55, 60, 65, 70, 75, 80, 85, 90, 95 or about 100 mm Hg. In most cases the optimal pressure window for treatment is about 73 mm Hg to about 88 mm Hg. In some target applications, a practitioner may establish a target pressure of about 80 mm Hg with +/−8 mm Hg variation.

The lumen for the method of treating abnormal uterine bleeding can contain a valve operable to open or close the lumen. A syringe or other injection means can also serve as the first valve.

The sclerotherapeutic agent for the method of treating abnormal uterine bleeding can be a vascular sclerosing agent. Sclerosants include detergents, such as sodium tetradecyl sulfate, polidocanol, sodium morrhuate and ethanolamine oleate; osmotic agents, such as hypertonic sodium chloride solution and sodium chloride solution with dextrose; and chemical irritants, such as chomated glycerin and polyiodinated iodine. The sclerotherapeutic agent preferably comprises polidocanol. The source of the treatment fluid for the method of treating abnormal uterine bleeding can further comprise a reservoir configured to hold and contain the treatment fluid (sclerotherapeutic agent) until it is to be administered. The reservoir [not shown] can be integral to the device, or separate and connected to it prior to dispensing the treatment fluid.

The method of treating abnormal uterine bleeding can further comprise administering a second therapeutic agent.

Still another aspect of the disclosure is directed to a device for treating abnormal uterine bleeding, where the device comprises a) a shaft having a proximal end and a distal end; and b) a lumen enclosed within the shaft having a port at the proximal end and a valve operable to open or close the lumen, for introducing a treatment fluid, and an opening at the distal end for dispensing the treatment fluid. Preferably, the device further comprises a conical gel tip on the shaft's distal end.

A further aspect of the disclosure is directed to a method of treating abnormal uterine bleeding, comprising the steps of 1) inserting the distal end of the above device for treating abnormal uttering bleeding into the uterus of a subject suffering from abnormal uterine bleeding, where the abnormal uterine bleeding is characterized by one or more bleeding blood vessels at or near a site of bleeding; 2) providing a source of treatment fluid, optionally comprising a sclerotherapeutic agent, to the port; and 3) introducing the treatment fluid via the port, through the lumen, out the distal shaft opening and into the uterus, at a positive pressure effective to provide retrograde flow of the treatment fluid into the one or more bleeding blood vessels. The intrauterine pressure is preferably maintained at about 10 to about 100 mm Hg, or about 10 to about 60 mm Hg, or about 15 to about 55 mm Hg, or about 20 to about 50 mm Hg, or about 25 to about 45 mm Hg, or about 30 to about 40 mm Hg, or about 30 to about 60 mm Hg, or about 30 to about 100 mm Hg, or about 50 to about 100 mm Hg. The intrauterine pressure can be about 10, 15, 20, 25, 30, 35, 40, 45, 50, 55, 60, 65, 70, 75, 80, 85, 90, 95 or about 100 mm Hg. In most cases the optimal pressure window for treatment is about 73 mm Hg to about 88 mm Hg. In some target applications, a practitioner may establish a target pressure of about 80 mm Hg with +/−8 mm Hg variation.

Another aspect of the disclosure is directed to a method for determining quantitative blood loss (QBL) in obstetric hemorrhage, comprising the steps of: 1) providing a device comprising a) a shaft having a proximal end and a distal end; b) a first lumen enclosed within the shaft having a first port at the proximal end for optionally introducing a treatment fluid, and a first opening at the distal end for dispensing the optional treatment fluid; c) a second lumen within the shaft having a second port at the proximal end and a second opening at the distal end for evacuating blood therethrough; d) a deployable sleeve at the distal end of the shaft; and e) an occlusive flange positioned between the proximal and distal ends of the shaft; 2) inserting the distal end of the device into the uterus of a subject suffering from obstetric hemorrhage; 3) deploying the deployable sleeve so that the sleeve is positioned against a sidewall of the uterus and seals the uterine cavity; 4) locking the device in place by positioning the flange against the fornix of the cervix; 5) evacuating blood through the second lumen within the shaft to a device configured to measure or weight the evacuated blood; and 6) optionally, introducing a treatment fluid, optionally comprising a sclerotherapeutic agent, through the first port. The device of the QBL method can be configured to measure or weight evacuated blood, comprising a receiving container, measuring container, weighing container, receiving means, measuring means, or weighing means. It shall be understood that in some implementations of the QBL method, the device may be inserted vaginally/transcervically solely for the purpose of measuring the active blood loss. That is to say that in some implementations no treatment fluid is required as the sole purpose is to quantify blood loss.

In those implementation is which a treatment fluid is applied, the source of the treatment fluid for the method for determining QBL in obstetric hemorrhage can be an injection means. The injection means can comprise a syringe and the treatment fluid does not necessarily require any particular drug but rather may be a sterile solution such as saline rather than a sclerotherapeutic agent. Nonetheless, a sclerotherapeutic agent may also be employed as the treatment fluid. The deployable sleeve for the device can be configured to radially expand the sleeve from the shaft when deployed. The deployable sleeve can comprise multiple anchors that peripherally expand to seal the uterine cavity. The deployable sleeve can be manually deployed or activated by pressure of the treatment fluid. The occlusive flange of the device can be slidable along the shaft. The second lumen of the device can be parallel to the first lumen. The second lumen of the device can surround the first lumen. The deployable sleeve of the device can further comprise an external membrane cover. The external cover can comprise a silicone rubber membrane. The multiple anchors of the device can deploy to seal the external cover within the uterine cavity. The first lumen of the device can contain a first valve operable to open or close the lumen. A syringe or other injection means can also serve as the first valve. The second lumen can contain a second valve operable to open or close the lumen. As explained above, the sclerotherapeutic agent for the QBL method may not require any particular drug although in some treatment modalities an administered drug may be a vascular sclerosing agent. The sclerotherapeutic agent can be a vascular sclerosing agent. The sclerotherapeutic agent can comprise polidocanol.

The QBL method can further comprise administering a second therapeutic agent.

### EXAMPLES

The following examples are intended to be illustrative of the present disclosure, and in no way limit its scope.

### Example 1. Device for Treating Post-Partum Bleeding

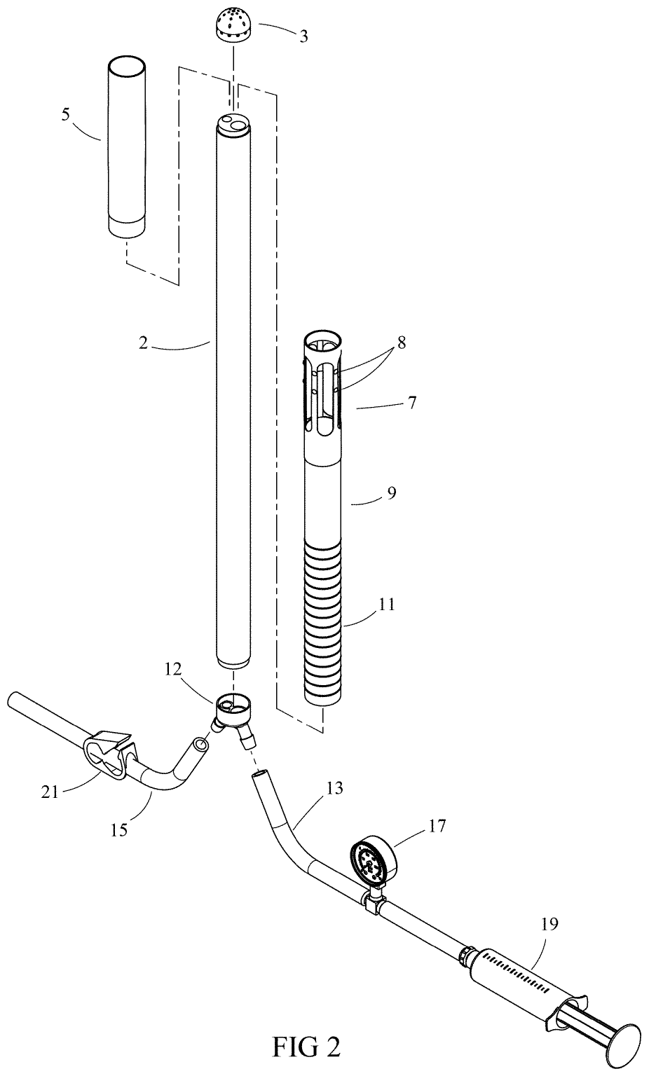

The device of FIG. 1 is assembled as shown in FIG. 2, and contains a shaft 2 containing one or more lumens, a distal endcap 3, an undeployed sleeve 5 including external cover, an undeployed occlusive flange 7 with its external cover, a slidable segment 9, a hand grip 11 on the slidable segment, a proximal endcap 12 with ports to lumens, an injection line 13 feeding into the first lumen within the shaft, a drain line 15 connecting to second lumen within the shaft, a pressure gauge 17, a syringe 19, and a pinch clamp 21.

### Example 2. Method of Treating Post-Partum Bleeding

The device of Example 1 is inserted into and positioned in a postpartum patient's bleeding uterus, as shown in FIG. 7. The deployable sleeve 5 is deployed against a sidewall of the uterus 110 forming the deployed sleeve 50 (FIG. 8), thereby sealing the interior of the uterus. Simultaneously the occlusive flange 7 is deployed to form 70 (FIG. 8) and positioned against the cervix 120, thereby locking the device in place. Thereafter, treatment solution is introduced by the syringe 19 through the injection line 13 into the first lumen of the shaft 2 and out through the first opening at the distal end through the endcap 3 and into the uterus (See FIG. 6A). The treatment solution is maintained under an appropriate pressure to provide retrograde flow of the solution into one or more bleeding blood vessels, as measured by pressure gauge 17. Intrauterine pressure is maintained at about 10 to about 100 mm Hg. Following treatment, excess treatment solution and any blood is evacuated back through the endcap 3, into the second lumen within shaft 2, to a drain line 15 for drainage and safe waste disposal.

### Example 3. Device for Treating Abnormal Uterine Bleeding

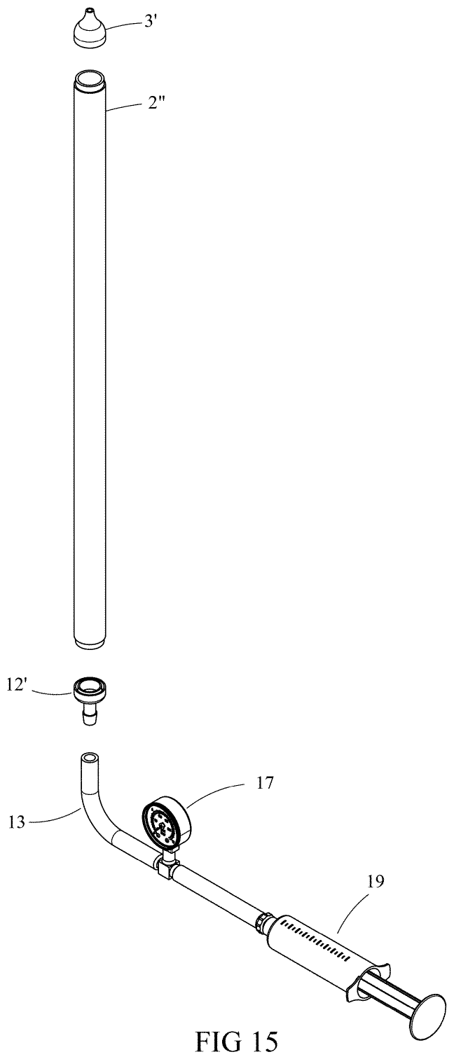

The AUB device of FIG. 14 is assembled as shown in FIG. 15, and contains a shaft 2″ containing a lumen, a distal conical gel tip 3′, a proximal endcap 12′ with a port to the lumen, an injection line 13 feeding into the lumen within the shaft, a pressure gauge 17, and a syringe 19.

### Example 4. Method of Treating Abnormal Uterine Bleeding (not Pregnancy Related)

The device of Example 3 is inserted into and positioned in the vagina of a patient suffering from abnormal uterine bleeding (non-pregnancy related), as shown in FIG. 17. Thus, the distal end of the AUB device 1′ is inserted into the vagina and positioned against the patient's external cervical os. Forward manual pressure from the practitioner applied through the shaft 2″ of the device seals the conical gel tip against the external cervical os to provide a pressure seal. Thereafter, treatment solution is introduced by the injection means 19, e.g., a syringe, through the injection line 13 into the lumen of shaft 2″ and out through the conical gel tip 3′, through the cervical canal and into the uterine cavity. The treatment solution is maintained under an appropriate pressure to provide retrograde flow of the solution into one or more bleeding blood vessels, the pressure being monitored by pressure gauge 17. The pressure should be maintained at about 10 to about 100 mm Hg. Following completion of treatment, the device is removed, and spontaneous drainage of the treatment solution and blood occurs.

### Additional Balloon Embodiments

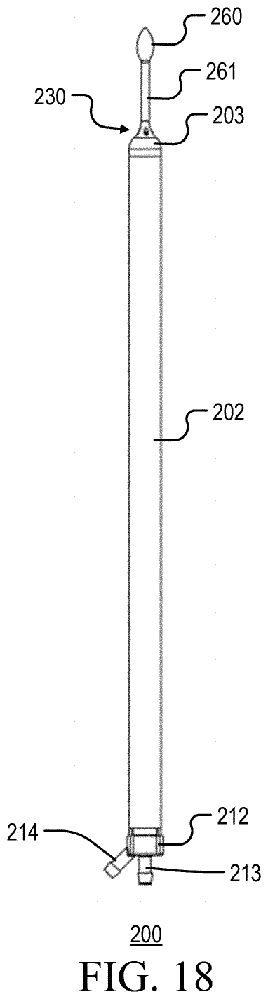

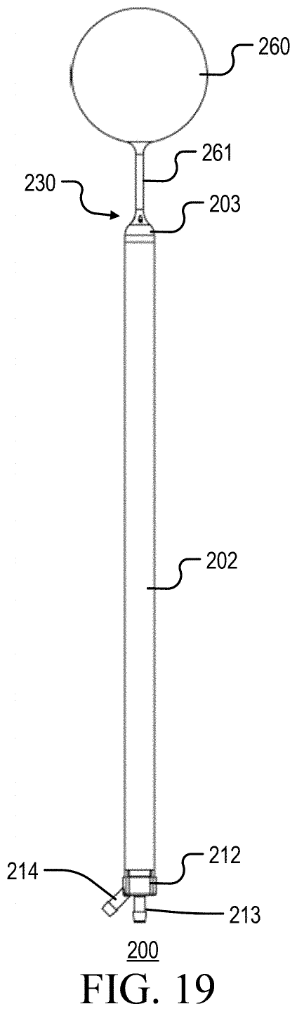

FIGS. 18 and 19 illustrate another embodiment of an AUB device 200. Device 200 may be similar to the PPH device previously discussed above and shown in FIGS. 14-16C. Device 200 may have the same, similar, and/or substantially the same features, components, and functionality as described above. Additionally, device 200 may further include a means for deploying a balloon 260 inside of a uterus 100 of the patient. Accordingly, duplicative description will be omitted or only briefly described and like numbering of parts will be maintained where feasible, e.g., the base device embodiment number 200 may be added to a previously described component having similar structural characteristics and/or functionality.

FIG. 18 illustrates an initial non-deployed configuration of the AUB device 200 and FIG. 19 illustrates a deployed configuration of the AUB device 200. Referring generally to FIGS. 18-19 an AUB device 200 may include a shaft member 202 taking a generally elongate cylindrical shape. In various embodiments, shaft member 202 may be a hollow tube that functions as a housing for various internal components as will be explained in further detail below. In one example, shaft member 2 may function as a housing for other components and/or a conduit for the delivery of treatment fluid to the patient's uterus or even for the evacuation of fluid from the patient's uterus. Shaft member 2 may include a distal endcap 203 having at least one lumen 230 at a distal end thereof and a proximal endcap 212 at a proximal end thereof. In this embodiment, distal endcap 203 is a flexible member having a conical shape that serves the function of forming a seal with the cervix and permitting treatment fluid to flow through the at least one lumen 230 through the cervical canal and into the uterine cavity. Distal endcap 203 may be formed of any suitable biocompatible material, e.g., a gel like material such as silicon or an elastomeric material or substance that has semi-fluid like properties. These material properties may facilitate and/or allow the endcap 203 to conform to varying anatomic shapes of the external cervical os (opening). This structure and material properties may function to form a fluid tight seal permitting introduction of the treatment fluid to the cavity within a desired pressure range and allow that pressure to be maintained throughout the duration of a treatment cycle, i.e., a span of time.

In other embodiments, multiple lumens 230 may be distributed around the outside surface of distal endcap 203. As illustrated, distal endcap 203 has a maximum width where it is coupled to the shaft member 2 that approximates a width of the shaft member 2. In other embodiments, distal endcap 203 may have a maximum width where it is coupled to the shaft member 2 that is greater than (or smaller than) a width of the shaft member (not illustrated). In this sense, it is contemplated that various embodiments in accordance with the principles disclosed herein may include a plurality of differently sized distal endcaps 203 that have a size and shape generally corresponding to a particular patient. For example, a young patient that has not delivered a child may require a relatively smaller distal endcap 203 relative to an older patient that has delivered a child.

In use, a practitioner may deliver a treatment fluid through the endcap 203 by any previously disclosed means through the proximal endcap 212. In this embodiment, proximal endcap 212 may include a first injection port 213 for the delivery of a treatment fluid into the uterine cavity and a second injection port 214 for the delivery of a fluid to the balloon 260. In various embodiments, the first injection port 213 may be used to deliver treatment fluid directly into an interior cavity of shaft 202 and out of the at least one lumen 230. Alternatively, the first injection port 213 may be used to deliver treatment fluid to an interior delivery channel that is in fluid communication with the at least one lumen 230.

Different from previously explained embodiments, a hollow tube 261 (may also be referred to as a pipe or channel) may extend through and/or from the distal endcap 203 and support an inflatable balloon 260 at a distal end thereof. The hollow tube 261 may be a flexible member having some tolerance for deforming during insertion. Alternatively, hollow tube 261 may be a thin and substantially rigid member. In this embodiment, the hollow tube 261 is in fluid communication with a second injection port 214. For example, an interior tube extending longitudinally through the shaft 202 may be coupled to the second injection port 214 and the hollow tube 261. In this way, a practitioner may inflate the balloon 260 by injecting a fluid through second injection port 214 by any of the previously disclosed injection means, e.g., a syringe or pump.

In at least one example method of use, a practitioner may use a syringe to inject a saline solution into the balloon 260. Additionally, a practitioner may take care to inject an appropriate amount of saline solution such that the balloon 260 expands a particular volume that takes into account the relative intrauterine cavity volume of the particular patient being treated. Furthermore, the balloon 260 may be injected before and/or after treatment fluid is injected through first injection port 213 but in all cases the balloon 260 will be inflated after it is positioned inside of the uterus. At least one advantage of this embodiment is that the inflated balloon 260 may reduce the amount of fluid required to fill the intrauterine cavity. For example, by reducing the void space within the uterine cavity a relatively smaller amount of treatment fluid should be required to fill the intrauterine cavity. This may be particularly helpful in reducing the associated costs of the target treatment fluid. In some embodiments, the balloon 260 may serve a non-therapeutic purpose. For example, the balloon 260 may be inflated just enough that it does not push against the uterine walls (or at least insubstantially touches a minimal portion of the uterine walls) and/or will insubstantially change the internal pressure of the treatment fluid against the uterine walls. In other embodiments, the balloon 260 may be used for a therapeutic purpose in that it may be inflated such that it does push against the uterine walls and directly impact the internal pressure of the treatment fluid against the uterine walls. In at least one therapeutic example method of use, a practitioner may selectively inflate and/or deflate the balloon 260 to help maintain an ideal internal pressure in the uterine cavity over time. In at least some cases, this example may be concurrently performed with the delivery of treatment fluid into the uterine cavity.

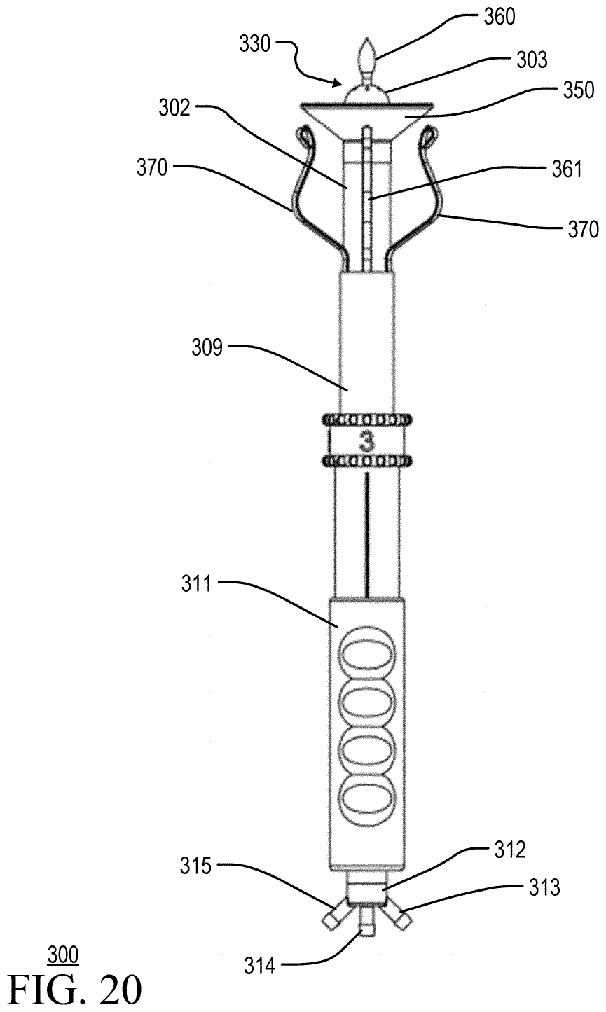

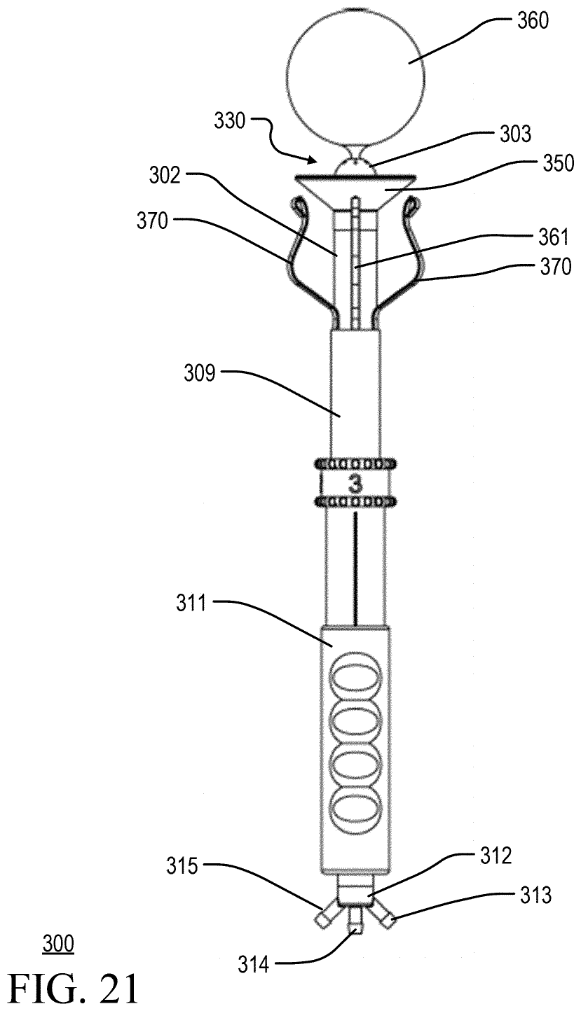

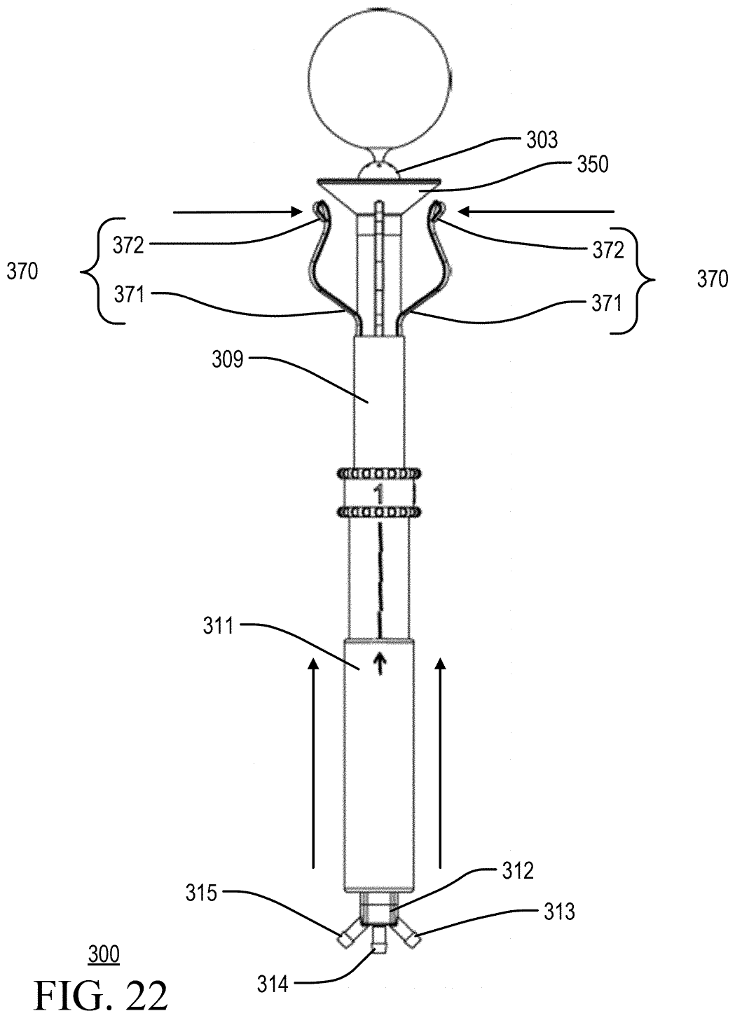

FIGS. 20-22 illustrate another embodiment of a PPH device 300. FIG. 20 shows a first illustration of a PPH device in a non-deployed configuration. FIG. 21 shows a second illustration of the PPH device of FIG. 19 in a deployed configuration. FIG. 22 shows another illustration schematically showing relative motion of a moveable handle of the device of FIGS. 20 and 21.

Device 300 may be similar to the PPH device previously discussed above and shown in FIGS. 1-6 and 9-13. Device 300 may also be similar the AUB device discussed above in FIGS. 18-19. Device 300 may have the same, similar, and/or substantially the same features, components, and functionality as described above. Additionally, device 300 may further include a means for deploying a balloon inside of a uterus 100 of the patient. Accordingly, duplicative description will be omitted or only briefly described and like numbering of parts will be maintained where feasible, e.g., the base embodiment number 300 may be added to a previously described component having similar functionality.

Referring generally to FIGS. 20-22, an alternate PPH treatment device 300 is disclosed. As illustrated, device 300 may contain an inner shaft 302 in which a distal endcap member 303 and a proximal endcap member 312 are coupled to. The inner shaft 302 may be a hollow tube that functions as a housing for various internal components as will be explained in further detail below. In one example, shaft member 302 may function as a housing for other components and a conduit for the delivery and/or removal of treatment fluid. Shaft member 302 may include a distal endcap 303 having at least one lumen 330 at a distal end thereof and a proximal endcap 312 at a proximal end thereof. In this embodiment, distal endcap 303 is a hemispherical shaped member having a plurality of lumens 330 for delivering treatment fluid to the patient. In some embodiments, (not illustrated) the distal endcap 303 may further include at least one lumen 330 for removing fluid from the patient. For example, fluid may be removed from the patient via a dedicated lumen 330 back through the hollow shaft member 302 and out of a discharge port 315. In various embodiments, fluid may be removed by applying suction pressure at the discharge port 315 by, e.g., a bulb, a vacuum, a syringe, etc. Applying a suction force or pressure to draw fluid away from a patient's uterus may be particularly advantageous in cases in which the patient may have an atonic uterus. At least one example of having a dedicated drainage means may be that a practitioner can observe the volume of blood loss and/or treatment fluid being evacuated by the device 300. As illustrated, distal endcap 303 has a maximum width where it is coupled to the shaft member 302 that allows for the distal endcap 303 to be inserted, at least partially, inside of a patients cervix and/or uterus (see FIG. 7).

In the example embodiment, a hollow tube 361 (may also be referred to as a pipe or channel) may extend from discharge port 315 to an inside surface of the deformable cup 350. In use, the deformable cup 350 may form a seal against the cervix of a patient. Deformable cup 350 may be made of any biocompatible material, e.g., a silicon or elastomeric material. In use, the hollow tube 361 may be used to evacuate fluid from a patient's uterus. Additionally, as explained previously, a clamp member 21 (see FIG. 2) may be used to prevent any unwanted discharge of fluid from the patient's uterus and then the clamp member 21 may be opened to facilitate the discharge of fluid from the patient's uterus. Alternatively, fluid may be removed from the patient via a dedicated lumen 330 back through a dedicated drainage line that is coupled to the discharge port 315 (not shown).

In use, a practitioner may deliver a treatment fluid to the lumens 320 of endcap 303 by any previously disclosed means, e.g., through a syringe 19 in fluid communication with the proximal endcap 212 (see FIG. 5). In this embodiment, proximal endcap 312 may include a first injection port 313 for the delivery of a treatment fluid into the uterine cavity, a second injection port 314 for the delivery of a fluid to the balloon 360, and a discharge port 315 for the selective removal of fluid from the patient's uterus. In various embodiments, the first injection port 313 may be used to deliver treatment fluid directly into an interior cavity of shaft 302 such that it is pressurized, and the fluid may be released into the patient's uterus out of the at least one lumen 330. Alternatively, the first injection port 313 may be used to deliver treatment fluid to an interior delivery channel and/or tubing that is in fluid communication with the distal endcap 303 and at least one lumen 330. In various embodiments, a balloon 360 may be coupled to a distal most point of the distal endcap 303. For example, a connection port or nipple may be provided on the distal endcap 303 that is suitable for connecting the balloon 360 thereto. In use, a practitioner may inflate the balloon 360 by injecting a fluid into the proximal endcap 312 at the second injection port 314, e.g., a saline solution or other appropriate liquid. For example, a dedicated injection line may extend through the hollow shaft 302 (not visible). Alternatively, a dedicated injection line may extend along the outside of the hollow shaft 302 and on the inside of an outer sleeve, e.g., moveable shaft 309.

Different from previously explained embodiments, a moveable outer shaft 309 may surround the hollow interior shaft 302. The moveable outer shaft 309 may have an inside diameter substantially approximating the outside diameter of the hollow interior shaft 302 while allowing for some tolerance such that the moveable outer shaft 309 may freely slide forward and backward in a longitudinal direction of the interior shaft 302. Consistent with the disclosure herein, the moveable outer shaft 309 may also have various integrated contouring to allow for a dedicated fluid delivery (to lumens 330 or to the balloon 360) or fluid evacuation line (from cup 350 and/or hollow tube 361 depending on the particular configuration. In the example embodiment, an interior tube for inflating the balloon may extend longitudinally through the interior shaft 302 and/or the moveable shaft 309. In this way, a practitioner may inflate the balloon 360 by injecting a fluid through second injection port 214 by any of the previously disclosed injection means (see syringe 19 in FIG. 5).

Referring to FIG. 22, the moveable shaft 309 may be moveable forward and backward in a longitudinal direction along the interior shaft 302 (see arrows in FIG. 22). The moveable shaft 309 may be configured to act against at least one flange 370, e.g., a deformable tang, finger, or tab. Although in the illustrated embodiment two flanges 370 are visible it shall be understood that various embodiments may include a plurality of flanges 370, e.g., two, three, four, five, etc. In various embodiments, flange 370 may be coupled to the moveable shaft 309 or to the interior shaft 302. In at least one embodiment, flange 370 is coupled to a medial portion of the interior shaft 302. In an open position where the moveable shaft 309 is positioned adjacent the proximal endcap 312 the flange 370 is in a neutral position where it does not contact the cup 350. In use, a practitioner may grip the ergonomic handle 311 (see FIG. 21) and slide the moveable shaft 309 forward towards the distal endcap 303. In doing so, the lower portion 371 of the corresponding flange 370 may be pinched and/or shifted towards the interior shaft 302. In doing so, the end portion 372 of the corresponding flange 370 may be brought inwards to directly contact the cup 350. For example, in a closed position, the moveable shaft 309 may be gripped by handle 311 and moved towards the distal endcap 303 such that it pinches the flange 370 (or flanges 370) thereby bringing the corresponding end portion 372 into direct contact with the cup 350. In this way, the moveable shaft 309 and handle 311 are configured to bring the at least one flange 370 into direct contact with the cup 350 such that the at least one flange 370 provides a support to the cup 350 thereby forming a pressure tight seal with the cervix of a patient.

In another embodiment, the at least one flange 370 may be directly coupled to an end portion of the moveable shaft 309. In this way, a practitioner may slide the moveable shaft 309 towards the distal endcap 303 such that the at least one flange 370 directly contacts the cup 350. In doing so, the at least one flange 370 may support the cup 350 such that it may form a pressure tight seal against the cervix of the patient.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modification and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by the context.

Although particular embodiments of the present disclosure have been described above in detail, it will be understood that this description is merely for purposes of illustration and the above description of the disclosure is not exhaustive. Specific features of the disclosure are shown in some drawings and not in others, and this is for convenience only and any feature may be combined with another in accordance with the disclosure. A number of variations and alternatives will be apparent to one having ordinary skill in the art. Such alternatives and variations are intended to be included within the scope of the claims. Particular features that are presented in dependent claims can be combined, and fall within the scope of the disclosure. The disclosure also encompasses embodiments as if dependent claims were alternatively written in a multiple dependent claim format with reference to other independent claims.

It should be understood that various aspects disclosed herein may be combined in different combinations than the combinations specifically presented in the description, accompanying drawings, and claims. For example, features, functionality, and components from one embodiment may be combined with another embodiment and vice versa unless the context clearly indicates otherwise. Similarly, features, functionality, and components may be omitted unless the context clearly indicates otherwise. It should also be understood that, depending on the example, certain acts or events of any of the processes or methods described herein may be performed in a different sequence, may be added, merged, or left out altogether (e.g., all described acts or events may not be necessary to carry out the techniques).

1. A method of treating post-partum bleeding, comprising the steps of:
providing a device comprising:
a) a shaft having a proximal end and a distal end;
b) a first lumen enclosed within the shaft having a first port at the proximal end for introducing a treatment fluid, and a first opening at the distal end for dispensing the treatment fluid;
c) a deployable sleeve at the distal end of the shaft; and
d) an occlusive flange positioned between the proximal and distal ends of the shaft;
providing a source of treatment fluid comprising a sclerotherapeutic agent to said first port;
inserting the distal end of the device into the uterus of a subject suffering from post-partum bleeding, wherein the post-partum bleeding is characterized by one or more bleeding blood vessels at or near a site of bleeding;
deploying the deployable sleeve so that the sleeve is positioned against a sidewall of the uterus thereby forming a fluid seal with the uterine cavity;
locking the device in place by positioning the flange against the fornix of the cervix; and
introducing the treatment fluid via the first port, through the first lumen, out the distal shaft opening and into the uterus, at a pressure effective to provide retrograde flow of the treatment fluid into the one or more bleeding blood vessels.

2. The method of claim 1, further comprising:
inflating a balloon inside of the uterus.

3. The method of claim 2, wherein the source of the treatment fluid is an injection means and the injection means comprises a syringe or a bag.

4. The method of claim 1, wherein the deployable sleeve is configured to radially expand the sleeve from the shaft when deployed.

5. The method of claim 4, wherein the deployable sleeve further comprises multiple anchors that peripherally expand to seal the uterine cavity.

6. The method of claim 1, wherein the deployable sleeve is manually deployed or activated by pressure of the treatment fluid.

7. The method of claim 1, wherein the occlusive flange is slidable along the shaft.

8. The method of claim 1, further comprising a second lumen within the shaft, having a second port at the proximal end and a second opening at the distal end for drainage.

9. The method of claim 1, wherein the shaft contains the second lumen within the shaft.

10. The method of claim 9, wherein the second lumen is parallel to the first lumen.

11. The method of claim 10, wherein the second lumen surrounds the first lumen.

12. The method of claim 5, wherein the deployable sleeve further comprises an external cover.

13. (canceled)

14. The method of claim 12, wherein the multiple anchors deploy to seal the external cover within the uterine cavity.

15. The method of claim 14, wherein intrauterine pressure of about 10 to about 100 mm Hg is maintained.

16. The method of claim 15, wherein intrauterine pressure of at least about 30 mm Hg is maintained.

17. The method of claim 1, wherein the first lumen further comprises a first valve operable to open or close the lumen.

18. The method of claim 9, wherein the second lumen further comprises a second valve operable to open or close the lumen.

19. The method of claim 1, wherein the sclerotherapeutic agent is a vascular sclerosing agent.

20. The method of claim 1, wherein the sclerotherapeutic agent comprises polidocanol.

21. The method of claim 1, wherein the source of treatment fluid further comprises a reservoir.

22. The method of claim 21, wherein the reservoir is integral to the device, or connected to it prior to dispensing the treatment fluid.

23. The method of claim 1, further comprising administering a second therapeutic agent.

24. A device for treating post-partum bleeding, comprising:
a) a shaft having a proximal end and a distal end;
b) a first lumen enclosed within the shaft having a first port and a first valve at the proximal end operable to open or close the lumen, for introducing a treatment fluid, and a first opening at the distal end for dispensing the treatment fluid;
c) a deployable sleeve at the distal end of the shaft, wherein the deployable sleeve is configured to insert through the cervix of a subject; and
d) an occlusive flange positioned between the proximal and distal ends of the shaft.

25. The device of claim 24, further comprising:
an inflatable balloon disposed at a distal end of the shaft;
a balloon inflating lumen for introducing a fluid into the inflatable balloon;
a second lumen within the shaft, having a second port at the proximal end and a second opening at the distal end for drainage.

26-35. (canceled)

36. A method of treating post-partum bleeding, comprising the steps of:
1) inserting the distal end of the device of claim 24 into the uterus of a subject suffering from post-partum bleeding, comprising one or more bleeding blood vessels;
2) providing a source of treatment fluid comprising a sclerotherapeutic agent to said first port;
3) deploying the deployable sleeve so that the sleeve is positioned against a sidewall of the uterus and retains the treatment fluid within the uterus;
4) locking the device in place by positioning the flange against the fornix of the cervix; and
5) introducing the treatment fluid via the first port, through the first lumen, out the distal shaft opening and into the uterus, at a pressure effective to provide retrograde flow of the treatment fluid into the one or more bleeding blood vessels at or near a site of bleeding.

37. (canceled)

38. A method of treating abnormal uterine bleeding, comprising the steps of:
1) providing a device comprising:
a) a shaft having a proximal end and a distal end; and
b) a lumen enclosed within the shaft having a port at the proximal end for introducing a treatment fluid, and an opening at the distal end for dispensing the treatment fluid;
2) providing a source of treatment fluid comprising a sclerotherapeutic agent to said port;
3) inserting the distal end of the device into the external cervical os of a subject suffering from abnormal uterine bleeding, wherein the abnormal uterine bleeding is characterized by one or more bleeding blood vessels at or near a site of bleeding; and
4) introducing the treatment fluid via the port, through the lumen, out the distal shaft opening and into the uterus, at a pressure effective to provide retrograde flow of the treatment fluid into the one or more bleeding blood vessels.

39-49. (canceled)

50. A device for treating abnormal uterine bleeding, comprising:
a) a shaft having a proximal end and a distal end; and
b) a lumen enclosed within the shaft having a port at the proximal end and a valve operable to open or close the lumen, for introducing a treatment fluid, and an opening at the distal end for dispensing the treatment fluid.

51. (canceled)

52. A method of treating abnormal uterine bleeding, comprising the steps of:
1) inserting the distal end of the device of claim 50 into the external cervical os of a subject suffering from abnormal uterine bleeding, wherein the abnormal uterine bleeding is characterized by one or more bleeding blood vessels at or near a site of bleeding;
2) providing a source of treatment fluid comprising a sclerotherapeutic agent to said port; and
3) introducing the treatment fluid via the port, through the lumen, out the distal shaft opening and into the uterus, at a pressure effective to provide retrograde flow of the treatment fluid into the one or more bleeding blood vessels.

53. A method for determining quantitative blood loss (QBL) in obstetric hemorrhage, comprising the steps of:
1) providing a device comprising:
a) a shaft having a proximal end and a distal end;
b) a first lumen enclosed within the shaft having a first port at the proximal end for optionally introducing a treatment fluid, and a first opening at the distal end for dispensing the optional treatment fluid;
c) a second lumen within the shaft having a second port at the proximal end and a second opening at the distal end for evacuating blood therethrough;
d) a deployable sleeve at the distal end of the shaft; and
e) an occlusive flange positioned between the proximal and distal ends of the shaft;
2) inserting the distal end of the device into the uterus of a subject suffering from obstetric hemorrhage;
3) deploying the deployable sleeve so that the sleeve is positioned against a sidewall of the uterus and seals the uterine cavity;
4) locking the device in place by positioning the flange against the fornix of the cervix;
5) evacuating blood through the second lumen within the shaft to a device configured to measure or weight the evacuated blood; and
6) optionally, introducing a treatment fluid comprising a sclerotherapeutic agent through the first port.

54-70. (canceled)
